(19) United States
(12) Patent Application Publication
(10) Pub. No.: US 2004/0161435 A1
(43) Pub. Date: Aug. 19, 2004

(54) Skin Firming Anti-Aging Cosmetic Mask Compositions

(76) Inventor: SHYAM K. GUPTA, SCOTTSDALE, AZ (US)

Correspondence Address: SHYAM K. GUPTA, BIODERM RESEARCH, 5221 E. WINDROSE DRIVE, SCOTTSDALE, AZ, 85254, US

(21) Appl. No.: 10/248,753

(22) Filed: Feb. 14, 2003

(51) Int. Cl. A61K 7/42; A61K 7/06; A61K 7/00; A61K 35/78

(52) U.S. Cl. 424/401; 424/74; 424/725; 424/59

(57) ABSTRACT

I have discovered cosmetic mask compositions suitable for face, neck, chin or body applications. These compositions synergistically combine at least one skin beneficial cosmetic or drug composition with at least one composition to promote excess fat reduction, cellulite control, or muscle toning benefits. The mask composition also contains at least one binder composition that binds with other beneficial ingredients by electrostatic, atomic, or ionic charges to synergistically enhance their topical site-specific benefits. These mask compositions are suitable for a variety of delivery system methods that include peel-off mask, leave-in mask, moisturizing mask, exfoliating mask, prosthetic mask, soaking mask, depilatory mask, foaming mask, rinse-off mask, sloughing mask, rub-off mask, two-phase mask, dual-chamber mask, and self-heating (heat releasing) mask.

### BACKGROUND OF INVENTION

The enhancement of physical appearance occupies greater focus in human life than nearly all other daily life-related concerns combined. There are far more consumer products available for the beautification of human body than for the treatment of human ailments. The improvement of body tone and appearance is a growing, multibillion-dollar industry encompassing cosmetic, nutraceutical, pharmaceutical, and physical therapy disciplines. The consumer attention is focused on newest miracle ingredient in age-defying, anti-wrinkle, body firming, varicose reducing, and slimming compositions. Of all such cosmetic beauty concerns, the enhancement of facial appearance occupies the greatest focus and concern for humans, especially for the female human. The selective treatment of face is thus of paramount importance to consumers. There are a variety of cosmetic delivery systems, such as lotions, creams, sprays, splashes, gels, sticks, and such that have been used to provide beneficial ingredients and compositions to facial areas. A cosmetic mask offers one of the most desirable delivery systems for the delivery of skin beneficial ingredients and compositions to facial skin. However, the development of multifunctional cosmetic masks has been very limited in the prior art.

With chronological age and chronic exposure to adverse environmental factors, the visual appearance, physical properties, and physiological functions of skin change in ways that are considered cosmetically undesirable. The most notable and obvious changes include the development of fine lines and wrinkles, loss of elasticity, increased sagging, loss of firmness, loss of color evenness (tone), coarse surface texture, and mottled pigmentation. Less obvious, but measurable changes which occur as skin ages or endure chronic environmental insult include a general reduction in cellular and tissue vitality, reduction in cell replication rates, reduced cutaneous blood flow, reduced moisture content, accumulated errors in structure and function, and a reduction in the skin's ability to remodel and repair itself. Many of the above alterations in appearance and function are caused by changes in the outer epidermal layer of the skin, while others are caused by changes in the lower dermis. Regardless of the stimulus for skin damage, when damage occurs, numerous natural and complex biochemical mechanisms are set into motion in attempts to repair the damage. When the epidermis is injured, the epidermal basal cells respond to the injury by dividing at a more frequent rate. This increase in replication rate results in a more rapid replacement of the damaged epidermis with a new epidermis and stratum corneum, a process referred to as “epidermal cell renewal”. Common examples of injuries which can increase epidermal cell renewal rates include abrasion, chemical damage, pH extremes, excessive sun exposure, or allergic or non-allergic contact irritation. If the injury is too severe, the increased replication will result in a “hyperplastic” epidermis and a thickened, poorly-functioning stratum corneum which is manifested as dry, rough scales. Other common stimuli which induce epidermal cell renewal include physical removal of the stratum corneum (i.e., an example of which is tape stripping, a process where tape is applied to the skin and pulled off, removing the top layer of the stratum corneum with it) and friction (i.e., on the soles and heels of the feet), all processes which result in epidermal hyperplasia. Hydroxy acids and retinoids also induce epidermal hyperplasia at appropriate concentrations, although the mechanisms appear to be different. It is believed by many that hydroxy acids exert their effects by inducing physical exfoliation of the corneum, whereas the retinoids more likely work by interacting with cytoplasmic and nuclear binding receptors to alter gene expression.

With age and chronic environmental exposure (notably UVA, UVB, and IR radiation), the dermis undergoes changes in structure and function which result in many of the characteristics of aged skin, including loss of elasticity, formation of wrinkles, loss of water-holding capacity, sagging, and poor microcirculation. At the molecular level, these changes have been correlated with biochemical changes in the content and structure of the extracellular matrix to which the major cells of the dermis (i.e., the fibroblasts) reside. Collagen becomes highly cross-linked and inelastic, elastin is reduced in amounts and is incorrectly distributed, and the glycosaminoglycans become reduced in amounts, which results in reduced intercellular water. As a result of this changed architecture, the normal amounts and distribution of trace metal ions, growth factors, hormones, and cytokines becomes altered which causes the fibroblasts to become metabolically less active or quiescent. Although these cells have natural mechanisms to repair themselves and the matrix in which they reside, with age and too much damage, they are less able to repair the damage, and the condition continues to deteriorate. If the quiescent fibroblasts can be metabolically activated and stimulated to divide, they will synthesize new extracellular matrix and the old, damaged matrix will be enzymatically degraded and replaced. This process of balanced synthesis and degradation is referred to as “dermal remodeling.” The activation process can be accomplished in many different ways, including chemical stimulation by selected hormones, growth factors, cytokines, vitamins, botanical extracts and retinoids, or by increasing the nutrient supply (i.e., blood flow) to the tissue. These problems have been described in detail in U.S. Pat. No. 6,495,126 (to Schiltz). However, Schiltz did not disclose any cosmetic compositions, especially any face or body mask compositions, to alleviate the problems of facial skin aging.

Excess weight and fear of obesity are the second most important concerns for mankind, next only to the fear of aging. A legionary number of compositions for slimming, fat reduction, and obesity control are available. Most weight loss dietary compositions, pharmaceuticals, and nutraceutical aids are designed to decrease the amount of body fat in an individual by decreasing the individual's appetite for food, decreasing the amount of food absorption in the individual, slowing down the rate of fatty acid synthesis within the body, or increasing the rate of catabolism of fatty acids. The control of body fat, and various biochemical mechanisms that can be utilized for the management of body fat, control of fatty tissue, maintenance of lean body mass, and slimming of body parts and body organs has been described in detail in U.S. Patent Application Number 20010041708 (to Yuan-Di Chang Halvorsen, et al.), U.S. Pat. No. 5,804,596 (to Majeed et al.), U.S. Patent Application number 200201 87943 (to Majeed et al.), and U.S. Patent Application Number 2002011 2253 (to S. J. Wakil et al.). The following are some examples of weight loss products and their mechanisms. Dexfenfluramine increases the brain levels of serotonin, a neurotransmitter and neurohormone that quell the appetite. Sibutramine also increases the levels of serotonin, as well as noradrenaline, and works to quell the appetite. Neuropeptide Y inhibitors curb the appetite, as well as stimulating the body to burn more sugars and less fat. Bromeriptine mimics the neurotransmitter dopamine, and may reduce blood sugar and fat production by the liver. Leptin, a hormone generated by adipocytes, affects the hypothalamus. Cholecystokinin, a hormone and neurotransmitter, acts to reduce appetite. Butabindide blocks an enzyme that inactivates cholecystokinin. Orlistat interferes with pancreatic lipase, which results in poor absorption of dietary fat. Insulinotropin is a glucagon-like hormone which prevents obesity by slowing down the emptying of the stomach. Bta-243 stimulates beta-adrenergic receptors on adipocytes, with a resulting increase in the burning of fatty acids. Troglitazone is a synthetic hormone which signals muscle cells to utilize fat for energy, rather than sugars. Cytokine regulators change the activity of hormone-like cytokines and alter the communication among cells, resulting in weight loss. Hydroxycitric acid acts as an inhibitor of enzyme citrate lyase, which subsequently slows down the synthesis of fatty acids and increases the rate at which fatty acids are burned.

The appearance of face being the most important concern for mankind, a combination of compositions that includes ingredients to reduce excess fat on face, reduce “double chin”, slim neck area, and to also provide antiaging and anti-wrinkle benefits has been of high consumer interest. A mask product that can be applied to face, chin, and neck areas to deliver the fat reducing, slimming, toning, antiaging, and anti-wrinkle compositions specifically on such site-specific locations of human body for their maximum absorption, bioavailability, and benefits has been in high consumer demand. Such compositions have been unavailable, as a review of prior art literature has established. Additionally, a mask is considered to have an adsorption effect for removing unwanted oils, pollutants, impurities, and dead cells from the stratum corneum. Removal of the mask is believed to assure deep cleansing of the skin, in particular of the horny layer of the epidermis. It also provides a state of hyper-hydration of the epidermis, resulting in an improvement in skin tone and texture. A scientifically designed mask can also provide skin and body beneficial ingredients for topical delivery for imparting their maximum absorption, bioavailability, and efficacy.

A number of cosmetic mask compositions have been disclosed in the prior art. For example, among the commercially available mask forming products is a clear gel from Revlon Corporation known as “Honey Masque”. The listed contents include water, ethyl alcohol, polyvinyl alcohol/vinyl acetate copolymer, dimethicone copolyol, propylene glycol, PEG-8, honey, oleth-10 phosphate, fragrance, preservative and colors. The dry-down time for the film to become non-tacky ranges from 15 to 30 minutes. A product sold by the Procter & Gamble Company under the trademark of “Noxzema.RTM. Deep Cleansing Mask” is based upon a polyvinyl alcohol film-forming material solubilized in ethanol; other ingredients include sorbitol, PEG-4 steareth-20, PEG-32, PEG-6, preservatives, fragrance, essential oils and colors. Dry-down time for this product is at least 15 minutes.

U.S. Patent Application 20020039976 (L. Sebillotte-Arnaud, et al.) discloses a rinsable, foaming, cleansing composition comprising at least one hydrophobic silica and at least one oxyalkylenated compound. Although this composition can be applied as a cosmetic mask, it provides only cleansing functions. This composition is thus of very limited application for any combination benefits.

U.S. Pat. No. 6,379,702 (Lorenz et al.) discloses a hydrophilic gel, which comprises a blend of acid-neutralized chitosan and a poly (N-vinyl lactam). The gel may be formed into a wound packing or cavity dressing where, unlike hydrocolloid dressings, it is able to absorb exudate without losing its gel structure. It can also be utilized as a drug carrier for transdermal devices and for use in dry skin masks to deliver moisturizers to the skin. It can also be used in prosthetic devices such as breast implants. Lorenz et al. only provide moisturization benefits in a facial mask composition, and do not include any antiaging or other facial skin enhancement ingredients.

U.S. Pat. No. 6,296,840 (Rodan) discloses a colored facial masque that is formed from a dry powder masque component and an activator liquid component. It is mixed prior to use. This mask is not convenient for consumers, as two separate products must be purchased, and mixing errors of the two-part composition can cause performance-related problems, since each phase contains critically important components for maximum benefits.

U.S. Pat. No. 5,747,022 (Slavtcheff) discloses a composition for forming a peelable skin mask. It is based on a combination of polyvinyl alcohol and a hydrophobically modified acrylate or methacrylate polymer. Most effective is a combination of two weight grades of polyvinyl alcohol, a low weight grade having a number average molecular weight ranging from about 15,000 to about 27,000 and a high weight grade having a number average molecular weight ranging from about 44,000 to about 65,000. The acrylate or methacrylate polymer preferably is a copolymer formed from a carboxylic monomer and a C.sub.8-C.sub.30 acrylate ester. This composition is thus limited to a specific delivery system (i.e. peel-off mask), which is dependent on a specific molecular weight range of a specific ingredient in a specific combination with another specific ingredient. It is thus of very limited utility.

U.S. Pat. No. 6,199,560 (North et al.) discloses a prosthetic cosmetic mask that includes a handle carrying a pad adjacent one end. The pad is formed of a plurality of leaves with a distal edge of each leaf being anatomically contoured with a concave zone to facilitate lateral placement against one's face beneath an eye, the lid of which is to receive makeup. This mask is thus a mechanical device to hold cosmetic ingredients in place on the face. It does not disclose any chemical compositions that may benefit the appearance of face.

U.S. Pat. No. 5,893,872 (LaFuente) discloses a prosthetic cosmetic mask having a specially sculpted body contact surface adapted to treat a subject's distinctive bodily characteristics. It is formed by first forming a negative impression of the subject's targeted body area, filling the impression with a hardening material, the hardening material being in the form of a mold reflecting a positive image of the body area. This mask is thus a mechanical device to hold cosmetic compositions in place on the face and other body parts. It does not disclose any chemical compositions that may benefit the appearance of face.

U.S. Pat. No. 5,720,949 (Davis) discloses a cosmetic mask product for application to the skin of the consumer, in particular, to the face of the consumer. The cosmetic mask product comprises a first composition containing an effervescent agent in a cosmetically suitable vehicle and a second composition containing an acid component in a cosmetically suitable vehicle, the first and second compositions being sequentially applied to the skin of the consumer in any convenient order of application. Upon application of the last of the two compositions, a foaming action occurs as a result of gas being liberated by virtue of the reaction between the effervescent agent and the acid component, with concomitant release of heat of reaction. After a time the residue is removed from the skin of the user by means of a scraper or cloth. This composition is not considered useful for the development of cosmetic masks that have multiple benefits combined and that are easy to apply. Moreover, such effervescent masks have limited consumer acceptance due to their difficulty of use and application.

U.S. Pat. No. 5,716,599 (Golz et al.) discloses a cosmetic preparation containing kaolin which may be employed as a mask, lotion, gel or cream with a non-sticky, non-plasticized consistency and with a content of white kaolin with a high proportion of kaolinite and spherical inorganic particles. This preparation with an inhibitive effect with regard to inflammation permits high kaolin contents to be used in masks and gels. A number of similar masks (called “Mudpacks”), which are based on various clays, are also well known in the prior art. Such mask compositions have vastly ignored to include antiaging, face slimming, and other beneficial ingredients in synergistic combinations. Moreover, such clay-based mask compositions have strong adsorptive power, which hinders with the transfer of any skin beneficial ingredients from the clay particles to skin.

U.S. Pat. No. 5,599,546 (Klein) discloses another “mudpack” mask composition that includes an acidic material selected from the group consisting of alphahydroxy acids, carboxylic acids, halocarboxylic acids, dicarboxylic acids, and combinations thereof, limonene-based oil, an absorbent carrier, and water. The compositions are applied to the skin as a facial mask and allowed to dry. The compositions both degrease the skin, and deliver therapeutic agents to the skin on a time release basis. These mask compositions are specific for carboxy acids that have pH-related problems. Such “mudpack” compositions are not suitable for the delivery of a variety of skin beneficial ingredients. Moreover, “mudpack” facial masks are now decreasing in their consumer acceptance.

U.S. Pat. No. 4,640,932 (Fong et al) describes a facial mask based on inorganic thickening agents, absorbent powders and/or organic gelling agents. Suitable as gelling agents are gelatin, starch, cellulose gums, guar gum, alginates and polyvinyl alcohols. Benzoyl peroxide is present as an active to control or at least mitigate acne vulgaris. Moreover, this product form is different, as it is intended to be immediately washed off. Neither does this type of product form a protective crust or rigid film, therebyrendering it impractical for use during the sleeping hours.

U.S. Pat. No. 5,139,771 (Gerstein) discloses a rinse-away face masque composition consisting of 1-70% of maltodextrin, about 0.1-15% of a seaweed derivative selected from the group consisting of salts or esters of alginic acid, carrageenan, and agar, and about 20-95% water. It does not contain any facially beneficial compositions for antiaging and slimming combinations, for example.

U.S. Pat. No. 5,026,552 (Gueret et al) discloses a mask formed from a mesh of woven fabric and a hydratable gel confined within holes of the mesh. Since the gel is confined, the mask can be pulled off all in one piece thereby performing a skin sloughing treatment.

U.S. Pat. No. 5,194,253 (Garrido et al) describes a method of forming a cosmetic treatment mask based on at least one hydrophilic film-forming polymer, ammonium hyaluronic acid, mineral or organic salt of deoxyribonucleic acid and water. Drying times with this technology are also relatively slow.

U.S. Pat. No. 4,014,995 (Juliano et al.) discloses cosmetic lotion, cream, and mask compositions based on oat flour. This disclosure is specific to oat flour and not applicable to compositions that contain several synergistically combined skin beneficial ingredients.

U.S. Pat. No. 5,158,772 (Davis) discloses topical compositions, including a mask, for application to the skin comprising a topically active agent which may be a cosmetic agent or a therapeutic agent, and a small, but carrier effective amount of a microbial polysaccharide polymer gel which is a beta-1,3 glucan-type polysaccharide. This disclosure mostly concerns the gel-forming properties of beta-1, 3-glucan, and, therefore, not of utility for multifunctional facial mask compositions.

U.S. Pat. No. 6,197,317 and U.S. Patent Application 20010004642 (Klein) disclose certain amino acids that have a very beneficial therapeutic effect on the skin. The compositions of Klein invention improve the tone and texture of the skin and decrease wrinkling due to drying and aging. The compositions also decrease photodamage to the skin such as photopigmentation and solar keratoses, and the compositions also operate to remove or decrease the severity of acne and seborrheic keratoses. Among the preferred amino acids are the alpha amino acids. Although such amino acids have been used in cosmetic mask compositions, Klein's disclosures are primarily for certain benefits of amino acids without any specific facial mask compositions that are unique.

U.S. Pat. No. 5,690,945 (Bui Bertrand) discloses gelled cosmetic composition for use as a cleansing mask, said composition comprising an aqueous gel and, as principal cleansing agent, graded spheroidal polyamide particles which are dispersed in said aqueous gel for skin exfoliation.

Chinese Patent CN1052426 (Z. Youan) discloses a cosmetic film mask made of pollen corpuscles, polyvinyl acetate and polymethacrylate, polyvinyl alcohol, and alginic sodium, polyethylene glycol and glycerol, and a surfactant such as sorbitol monolaurate. It is a cleansing mask.

Rumanian Patents RU RU2185147 and RU2184530 (M. S. Mikhajlova) disclose cosmetic masks with oat powder (oat flour), baking yeast, and wheat germ flakes. These skin nourishing mask compositions do not disclose any synergistic or combination benefits.

European Patent EP1 1 86291 (Potin et al.) discloses a cosmetic mask composition comprising water, polyvinyl alcohol, at least one copolymer of vinyl pyrrolidone and at least one oil. It is a cleansing mask. No synergistic benefits are claimed.

Canadian Patent CA2202735 discloses a mask product comprising first and second compositions for sequential application to the face of a consumer, one of said composition containing an effervescent agent and the other of said composition containing an acid component. Such two-part mask compositions are not convenient to many consumers, and they also do not provide any synergistic benefits to treat facial ailments or problems.

As can be noted from the above prior art citations, the preparation of most cosmetic mask compositions is not difficult, as it mostly requires a liquid component (such as water or a water-soluble organic solvent), one or two cosmetically or pharmaceutically beneficial ingredients, and a binder agent that mostly acts as a “glue” to bind the other components of the mask composition. It is well known that most such binder ingredients or compositions can be anionic, cationic, non-ionic, or amphoteric in their electrical charge properties. Such electrical charge properties can be used advantageously for synergistic topical delivery of skin and body beneficial ingredients or compositions. In that manner, the binder agent can perform a dual role. It acts to bind various ingredients or compositions of a mask, acting as a “glue”. It also acts to bring the components of the composition together due to such electric, atomic, or ionic charge properties.

It is the surprising discovery of the present invention that such electric charge properties of such binder agents or compositions synergistically enhance the skin or body beneficial properties of such skin or body beneficial ingredients or compositions. For example, chitosan has several free amino groups in its chemical structure. Such amino groups have a free pair of electrons on its nitrogen atoms that can be bound with electron accepting skin or body beneficial ingredients or compositions. By adding an acidic ingredient to chitosan, for example, the same amino groups become positively (i.e. cationically) charged, now capable of synergistically binding with anionic skin or body beneficial ingredients. In another example, alginic acid has several carboxyl groups in its chemical structure. These acidic groups can synergistically bind with amino groups of skin or body beneficial ingredients. Psyllium has several non-ionic hydroxyl groups in its chemical structure. This can synergistically bind with non-ionic groups of skin or body beneficial ingredients or compositions. As can be appreciated by those versed in this art that a mixture of such binder compositions may be necessary for synergistic topical delivery of skin or body beneficial ingredients or compositions that may have a mixture of anionic, cationic, amphoteric, or non-ionically charged chemical structures. In addition, in the above example, both chitosan and alginic acid provide an additional skin or body beneficial function. Chitosan is known to absorb fat, possibly helping facial skin slimming. Alginic acid provides deep moisturizing of skin. Colloidal oat meal, oat flour, and other proteinaceous compositions contain both acidic and amino groups (i.e. amphoteric charges). Most clays and zeolites have anionic charge. Even the non-ionic ingredients, for example starches, celluloses, natural gums, and such have hydroxyl groups, which can bind with other non-ionic ingredients via hydrogen-bonding or Van der Waal's forces. Such electrical binding properties of commonly used mask ingredients have not been recognized in the prior art.

Surprisingly, the compositions of the present invention can thus provide multiple synergistic benefits, based both on the proper selection of the binder agents based on the electrical charge properties of such binder agents or compositions, and the proper selection of skin and body beneficial ingredients or compositions that can synergistically bind by electrostatic forces with such binder agents or compositions for various masks applications. To illustrate this further, chitosan, a binder agent which contains free amino groups, when mixed with a fat-burning ingredient such as Hydroxycitric acid, and a collagen-boosting antiaging ingredient such as ascorbic acid, becomes ionized from the carboxyl groups of both ascorbic acid and Hydroxycitric acid and binds by electrostatic bonds with both such acidic ingredients. When such a composition is applied to facial skin, for example, it is known that human skin is acidic in nature and that acidity of the skin then binds with the amino groups of chitosan. Chitosan, which was bound by electrostatic bonds with both ascorbic acid and hydroxycitric acid, then releases both of those ingredients in close proximity of each other on the facial skin for both of them to provide their individual benefits in a synergistically enhanced manner. In another example, when alginic acid, a binder agent, is combined with another acidic ingredient, such as Hydroxycitric acid or ascorbic acid, or both, then virtually nothing happens between them. However, if calcium ions are also present, then calcium ions combine at one end with alginic acid carboxyl groups and the carboxyl groups of ascorbic acid or Hydroxycitric acid, or both, at the other end, since calcium is a divalent cation. Alginic acid thus becomes gelatinous, acting as a binding agent. It also holds both ascorbic and Hydroxycitric acids in close proximity of each other via electrostatic bonds. When such a composition is applied to skin, then the acidity of skin surface reacts with ionized alginic acid, converting it into non-ionized alginic acid, resulting in the deposition of both ascorbic acid and Hydroxycitric acid on the skin surface in close proximity of each other for their synergistic benefits. As can be appreciated by anyone skilled in the art, that amphoteric materials such as colloidal oat protein can bind by electrostatic bonds with both anionic and cationic materials. It is believed that the above illustrations provide a clear view of the complex chemistry of the innovative delivery systems of the present invention.

This invention relates to facial mask compositions that contain ingredients and compositions to provide facial area slimming, firming, cellulite reduction, or fat-reduction benefits. Additionally, such benefits are combined synergistically with ingredients and compositions for the treatment and regulation of topical disorders of facial area skin, such as skin aging, wrinkles, acne, rosacea, age-spots, striae distensae (stretch marks), pimples, skin infections and lesions, varicose veins, venous insufficiency, and skin redness.

In a further respect, this invention relates to facial and body mask compositions that are not limited to any specific single ingredient.

In a further respect, this invention relates to facial and body mask compositions that are not sensitive to any anionic, cationic, non-ionic, zwitterionic, or amphoteric ingredients or compositions.

In a further respect, this invention relates to facial or body mask compositions that are not limited to any specific pH, viscosity, molecular weight ranges, or mixing, or storage conditions.

In a further respect, this invention relates to facial or body mask compositions that are adaptable for a wide variety of mask delivery system methodologies that includes peel-off mask, leave-in mask, moisturizing mask, exfoliating mask, prosthetic mask, soaking mask, depilatory mask, foaming mask, rinse-off mask, sloughing mask, rub-off mask, self-heating (heat releasing) mask, two-compartment mask, and such.

In a further respect, this invention relates to facial and body masks compositions that provide synergistic combination of benefits. Such synergistic combinations are not limited to any specific compositions or combinations.

In a further respect, this invention relates to facial and body mask compositions that are faster drying without requiring the use of low molecular weight alcohols or volatile silicones in their composition.

### SUMMARY OF INVENTION

The present invention discloses cosmetic mask compositions suitable for face, neck, chin and other selective, local body applications. These compositions synergistically combine at least one skin beneficial cosmetic or drug composition with at least one composition to promote excess fat reduction, cellulite control, or muscle and skin toning benefits. The mask composition also contains at least one binder composition that binds with other beneficial ingredients by electrostatic, atomic, or ionic charges to synergistically enhance their benefits. These mask compositions are suitable for a variety of delivery system methods, such as peel-off mask, leave-in mask, moisturizing mask, exfoliating mask, prosthetic mask, soaking mask, depilatory mask, foaming mask, rinse-off mask, scrubbing mask, rub-off mask, two-phase mask, two-compartment (dual chamber) mask, and self-heating (heat releasing) mask.

### DETAILED DESCRIPTION

The reduction of the signs of skin aging, reduction of skin wrinkles, and reduction of age-spots occupies greater emphasis for the mankind now than ever before in human history. The cosmetic or drug compositions that provide treatment of skin aging, skin wrinkles reduction, skin exfoliating, treatment of acne, treatment of rosacea, age-spots reduction, skin surface whitening, skin surface brightening striae distensae (stretch marks) reduction, treatment of pimples, treatment of skin infections and lesions, varicose and spider veins reduction, blood microcirculation improvement, UVA/UVB protection of skin, and skin redness reduction can be selected from a great number of ingredients and compositions, some of which are already well known in the prior art. However, many of such ingredients or compositions known in the prior art have not been disclosed in any combination, and especially in any synergistically enhanced compositions that provide superior combination benefits in a mask delivery system.

Excess weight and fear of obesity are the second most important concerns for the mankind, next only to the fear of aging. Most weight loss dietary compositions, pharmaceuticals, and nutraceutical aids are designed to decrease the amount of body fat in an individual by decreasing the individual's appetite for food, decreasing the amount of food absorption in the individual, slowing down the rate of fatty acid synthesis within the body, or increasing the rate of catabolism of fatty acids. Moreover, such dietary compositions are not site-specific for any selective body part, such as face, chin, or neck.

Appearance of face being the most important concern for the mankind, a combination of compositions that includes ingredients to reduce excess fat on face, reduce “double chin”, slim neck area, and also provide antiaging and anti-wrinkle benefits has been of high consumer interest. The present invention discloses mask compositions that can be applied on face, chin, and neck areas for their maximum absorption and bioavailability of beneficial ingredients to provide site-selective slimming, toning, and excess fat reduction benefits that are synergistically combined with antiaging, anti-wrinkle, and similar facial surface enhancement benefits.

Most cosmetic mask compositions are based on a single or a combination of just a few ingredients that are suitable for a specific mask type, such as peel-off, rinse-off, rub-off, foaming and cleansing, and such. Usually such compositions are designed as masks for a single, specific purpose, and then additional ingredients are added to provide certain benefits or product claims. It is the purpose of the present invention to provide a system of designing the final composition of a mask based on first selecting the ingredients that are required in a synergistic combination to provide certain skin beneficial attributes. The selection of appropriate additional ingredients to finalize the mask composition for a specific delivery mode is then made. For example, to deliver face slimming and antiaging benefits in a peel-off mask or a deep moisturizing mask, the selection of slimming and antiaging ingredients in a synergistic combination is made first. The additional ingredients to make, for example, the peel-off mask or the deep moisturizing mask, are then selected to provide an overall combination of synergistically acting mask composition. Some of these points will become clearer in the Examples section of the present invention.

Facial and body mask compositions of the present invention can be designed in a variety of delivery system forms that includes peel-off mask, leave-in mask, moisturizing mask, exfoliating mask, “fillers” for prosthetic mask, soaking mask, depilatory mask, foaming mask, rinse-off mask, sloughing mask, rub-off mask, two-phase mask, self-heating (heat releasing) mask, and so forth.

A peel-off mask is applied as a liquid film that is thinly spread with fingers on the face or body part. It is allowed to dry for several minutes, then pulled away from face with fingers. It peels-off as a thin plasticized film. It is usually preferred that such masks require a relatively short period of time to dry down to be pulled-off. Such peel-off masks usually provide deep pore cleansing and skin debris removal functions.

A leave-in mask is applied as a liquid, lotion, or cream composition on the face or body part. It is allowed to soak in. It is not rinsed-off or removed by other means. Such masks usually provide skin nutritive and treatment ingredients, such as anti-acne, antibacterial, vitamins, and such that are absorbed with high bioavailability. Various moisturizing masks are generally designed as leave-in mask compositions.

A prosthetic mask is a physical device, such as molded plastic or plaster, that is filled or loaded with a liquid, lotion, cream, paste, or powder “filler” composition and applied to face or body part. It is then left in place for a prescribed period of time. Prosthetic device is then removed and face or body parts rinsed-off.

Exfoliating mask compositions can contain a chemical exfoliator or a physical exfoliator. The examples of chemical exfoliator include various hydroxy acids, fruit acids, and enzymes. The examples of physical exfoliators include various crushed nutshells, luffa particles, sand, polyethylene beads, wax beads, seeds, and such.

A rub-off mask is usually supplied as a paste that is applied to face or body parts and allowed to partially soak in. It is then rubbed with fingers or hand to remove mask compositions that have not soaked into the skin. This mask delivery system thus provides a combination of skin nutritive or treatment and mild skin exfoliation functions.

A foaming mask is applied to pre-wetted face or body part as a liquid or paste, then rubbed gently with fingers to generate foam. After a few minutes the foam is rinsed off. Such masks thus provide a cleansing and mild exfoliating function. Since the contact time with skin is relatively short, such masks generally do not provide adequate nutritive or treatment benefits.

A soaking mask is a liquid or thin lotion composition that is first soaked onto a piece of fabric or paper. Such fabric or paper pieces may be pre-cut to a shape, such as in the form of a face with holes for nose, lips, and eyes. After soaking, such pieces of fabric or paper are placed on the face and allowed to soak in for several minutes. The fabric or paper piece is then removed. The face may or may not require rinsing at this stage, depending on the composition that was soaked onto the fabric or paper.

Depilatory mask-is applied to face or body part for the removal of excess or unwanted hair.

A sloughing mask is usually a combination of foaming mask and exfoliating mask compositions to provide dual, combination benefit.

A two-phase mask is composed of two components, a powder or paste and a thin liquid or activator fluid. These two components are packaged separately. The two components are mixed immediately preceding their application. Although such two-phase compositions are beneficial, their lack of convenience (need to purchase two separate products) makes them unattractive to many consumers. A two-phase mask product that requires mixing of one composition only with some water would be more convenient to such consumers. Such two-phase mask product could thus be marketed as a liquid or paste, which is then combined with water as the “activator” second phase just before product application.

Another example of a two-phase mask is compositions that are packaged in a two-compartment packaging forms. Such dual-chamber delivery systems (such as U.S. Pat. No. 6,462,025; Vishnupad and U.S. Pat. No. 6,448,233; LaFevre et al.) have been disclosed.

A self-heating mask releases heat upon application to skin. This is because such masks release heat of hydration when they absorb water from skin surface. The water from skin surface can be from skin that was pre-wetted prior to mask application, or from evaporation of water from skin surface due to natural perspiration.

All of the above delivery systems for mask compositions can be prepared according to the present invention. The cosmetic mask compositions of the present invention suitable for facial or body application in various delivery system forms comprise: (1) at least one skin beneficial cosmetic or drug composition that provides treatment of skin aging, skin wrinkles reduction, skin exfoliating, treatment of acne, treatment of rosacea, age-spots reduction, skin surface whitening, skin surface brightening striae distensae (stretch marks) reduction, treatment of pimples, treatment of skin infections and lesions, varicose and spider veins reduction, blood microcirculation improvement, UVA/UVB protection of skin, skin redness reduction, or combinations thereof, and (2) at least one topical nutraceutical composition to promote excess fat reduction, cellulite control, or muscle toning, or combinations thereof, and (3) at least one binder composition that binds with the ingredients of the compositions in (1) and (2) by electrostatic, atomic, or ionic charges to synergistically enhance the benefits of (1) and (2), and (4) a cosmetically or pharmaceutically acceptable delivery system, or a carrier base composition.

The cosmetic or drug compositions that provide treatment of skin aging, skin wrinkles reduction, skin exfoliating, treatment of acne, treatment of rosacea, age-spots reduction, skin surface whitening, skin surface brightening striae distensae (stretch marks) reduction, treatment of pimples, treatment of skin infections and lesions, varicose and spider veins reduction, blood microcirculation improvement, UVA/UVB protection of skin, and skin redness reduction can be selected from a great number of ingredients and compositions, some of which are already well known in the prior art. However, many of such ingredients or compositions known in the prior art have not been disclosed in any combination, and especially in any synergistically enhanced compositions that provide superior combination benefits. Such compositions are further detailed in the claims section of the present invention.

It is a well known problem that the weight loss, slimming, or fat reduction usually causes skin wrinkles, as the skin takes much longer to adjust to the shrinkage of tissue from such fat reduction actions. A combination of weight loss, slimming, or other such fat reduction compositions with antiaging and wrinkle reduction benefits is thus very difficult, since such weight or fat reduction compositions may also cause skin wrinkle formation. It has been surprisingly discovered in the present invention that the efficacy of a combination of compositions or ingredients for the skin whitening, skin brightening, and reduction of age-spots and body fat reduction (body slimming) ingredients is synergistically enhanced by the inclusion of a composition that promotes the formation of collagen and elastin in the skin. Since many of such collagen and elastin boosting compositions also possess antioxidant and anti-inflammatory affects, it has also been found that such compositions also synergistically enhance other antiaging and wrinkle reduction compositions present in combination with weight loss, slimming, or fat reduction compositions. As is also evident from these findings, many such compositions that have antioxidant or anti-inflammatory affects may provide a dual benefit in the compositions of the present invention. Although not bound by any theory, it appears that the initial skin wrinkles that are first formed by the affects of body part slimming or fat reduction compositions are visibly reduced by enhanced collagen and elastin formation in the skin. This permits skin to then naturally adjust itself by shedding excess protein and other tissue thus reducing such initially formed wrinkles, at which point external body part or organ appears slim and toned. Such combinations of compositions are further detailed in the claims section of the present invention.

The binder component of the present invention serves multiple functions as follows. (I) It acts to bind the skin beneficial cosmetic or drug composition with composition to promote excess fat reduction, cellulite control, or muscle toning and keeps them in close proximity of each other for synergistic benefits by electrostatic, atomic or ionic charges (II) It acts as a rheology modifier to control the viscosity and flow properties that are required for various delivery system forms of mask compositions mentioned above. (III) It provides adhesion to skin for various mask delivery systems that require adhesion properties for such mask compositions, and, (IV) it provides skin feel, skin surface smoothing, skin purifying, skin surface exfoliating, and skin moisturizing benefits, such benefits are additional benefits from other ingredients of mask compositions.

Relative to the amounts of various ingredients mentioned in (1), (2), and (3) above, it is to be appreciated by anyone skilled in the art, that various ingredients have their own range of beneficial use based on their own generally recognized safety, efficacy, and consumer requirement criteria. Frequently such limits are not known until a composition has been tested for various criteria such as performance, benefits, stability, and cost, to mention a few. For practical reasons, the limits of use for such ingredients or compositions can be from about 0.0001% to about 90% for any single ingredient in a composition. For example, a mask composition may contain as little as 0.0001% of an antiaging antioxidant ingredient, and up to 90% of water as a carrier base, among other ingredients of that same composition. Similarly, another mask composition may contain up to 90% of glycerin as the carrier base, and several other compositions per (1), (2), and (3) above to make up for the balance. For these reasons, it is proposed that the range of various ingredients per (1), (2), and (3) above is to be set at from about 0.0001% to about 90% by weight basis for any single ingredient or composition. The balance amount can be from the composition in (4) mentioned above.

The cosmetically acceptable delivery system or a carrier base can be selected in the form of a lotion, cream, gel, spray, thin liquid, body splash, mask, serum, solid cosmetic stick, lip balm, shampoo, liquid soap, bar soap, bath oil, cologne, hair conditioner, salve, collodion, impregnated patch, impregnated strip, skin surface implant, and any other such cosmetically or pharmaceutically acceptable topical delivery forms. The cosmetically acceptable delivery system can be prepared separately and other necessary ingredients or compositions can be added to it separately in the form of mixtures, solutions, suspensions, or emulsions to facilitate the manufacturing of such compositions.

### EXAMPLES

The following examples are presented to illustrate presently preferred practice thereof. As illustrations they are not intended to limit the scope of the invention. All quantities are in weight %.

### Example 1

Chitosan Facial Mask Composition for Rinse-off Delivery System Including the Ingredients for the Reduction of Wrinkles and Excess Fat on face and Neck.

Ingredient % (1) Chitosan 5.0 (2) Lactic Acid 5.0 (3) Glycerin 18.0 (4) Water 65.8 (S) Hydroxycitric acid 5.0 (6) Niacinamide 0.5 (7) Glutathione 0.2 (8) Preservatives 0.5 Procedure: Mix 1, 2, and 3 to a paste. Mix 4 to 8 separately to a clear solution. Add this to main batch and mix. A clear gel product is obtained. It is applied on the face and neck and left for 10 to 30 minutes, then rinsed off.

### Example 2

Psyllium Facial Mask For Exfoliating Delivery System Including the Ingredients for Antiaging and Double Chin Reduction.

Ingredient % (1) Psyllium husk powder 10.0 (2) PEG-630.0 (3) Forskohlin 1.0 (4) Calcium Sulfate 5.0 (5) Soybean Fibers 5.0 (6) Oat Protein 5.0 (7) Carnosine 0.5 (8) Water 43.0 (9) Preservatives 0.5 Procedure. Mix 1 to 6 to a paste. Mix separately 7 to 9. Add to main batch and mix to a paste. A thin dough-like product is obtained. It is applied on the face and neck area with fingers. After 10 to 30 minutes, it is rubbed with fingers to remove most of the mask components, then rinsed off.

### Example 3

Chitosan Facial Mask for Prosthetic Delivery Systems with Ingredients for Skin Whitening and Reduction of Fatty Tissue in Puffy Eyelids.

Ingredient % (1) Chitosan 5.0 (2) Lactic Acid 5.0 (3) PEG-620.0 (4) Forskohlin (water soluble) 1.0 (5) Tetrahydrocurcumin 0.1 (6) Licorice Root Extract 0.5 (7) Water 50.9 (8) Hydroxycitric acid 5.0 (9) Hydroquinone 2.0 (10) Soybean Fibers 5.0 (11) Oat Protein 5.0 (12) Preservatives 0.5 Procedure. Mix 1 to 6 to a paste. Mix separately 7 to 12. Add to main batch and mix to a paste. It is applied to the prosthetic device, which is then placed on the face or other body area. After 10 to 30 minutes, prosthetic device is removed, and the mask residue not yet absorbed into the skin is washed off.

### Example 4

Sloughing Mask. This mask is rubber after application to slough-off dead skin cells and rejuvenate fresh skin cells. It is also an exfoliating or Rub-off mask delivery system.

Ingredient % (1) Paraffin wax 25.0 (2) Cetyl alcohol 1.0 (3) Propyl paraben 0.1 (4) Methyl paraben 0.2 (5) GMS-SE 4.0 (6) Stearic acid 3.0 (7) Emulsifying wax 5.0 (8) Deionized water 48.8 (9) Menthol 0.3 (10) Aloe Vera 0.5 (11) Corn starch 10.0 (12) Polydimethylsiloxane 2.0 Procedure: All ingredients are mixed and heated at 60 to 70 C. The mixture is cooled to room temperature. A thick paste is obtained. It is applied on face or body part with fingers. After 10 to 15 minutes, it is rubbed-off with fingers. It comes off as granular particles that contain impurities, dead skin cells, and body oil that has been removed from the body part.

### Example 5

Foaming Facial Mask with Ingredients for Deep Cleansing, Rosacea reducing and Excess Facial Oil Removing Effects.

Ingredient % (1) Chitosan 5.0 (2) Citric acid 5.0 (3) Glycerin 18.0 (4) Water 58.8 (5) Sodium Cocoyl Isethionate 10.0 (6) Polydimethylsiloxane 2.0 (7) Emblica (Phyllanthus emblica) extract 0.2 (8) Horse Chestnut extract 0.5 (9) Preservatives 0.5 Procedure: Mix 1, 2, and 3 to a paste. Mix 4 to 9 separately to a clear solution. Add this to main batch and mix. A thick, ttanslucent gel is obtained. It is applied on the face and neck and left for 10 to 30 minutes, then rinsed off. During rinsing, much foam is generated that provides cleansing action.

### Example 6

A Peel-off Mask Composition with Anti-wrinkle and Antiaging.

Ingredient % (1) Deionized Water 69.2 (2) Polyvinyl Alcohol 11.0 (3) Polyethylene Glycol 4.0 (4) Oleth-20 1.0 (5) Glycerin 1.5 (6) Sodium Hyaluronate 0.1 (7) Preservative 0.5 (8) Witch Hazel Extract 5.0 (9) Vitamin A Palmitate 0.1 (10) Vitamin E Acetate 0.1 (11) Niacinamide 3.0 (12) Ascorbic Acid 4.5 Procedure: Water was heated at 70 to 80 C. All ingredients were added with mixing. The product was cooled to give a translucent yellow syrupy gel. The product is applied to face and neck areas as a thin film. After 10 to 15 minutes, it is peeled-off with fingers.

### Example 6

An Anhydrous face Mask Composition with Heat-releasing Effect

Ingredient %1. Calcium Sulfate (Anhydrous) 30.0 (2). Glycerin 49.0 (3). Sodium Potassium Aluminosilicate 20.0 (4). Xanthan Gum 1.0 Procedure: All ingredients are mixed in a dry atmosphere. A white paste is obtained. The face is rinsed with water first, then the mask composition is applied as a film. The heat is felt immediately. It is rinsed off after 10 minutes.

### Example 7

A Clay-based Mask Composition with Astringent and Slimming Skin Beneficial Ingredients in a more Bioavailable form than common “Mud pack”Masks.

Ingredient % (1). Water 49.7 (2). Methyl Paraben 0.2 (3). Propyl Paraben 0.1 (4). Safflower Oil 6.0 (5). GMS-SE 4.0 (6). Vitamin E Acetate 0.1 (7). Plant Extracts 0.1 (8). Titanium Dioxide 2.0 (9). Glycerin 2.0 (10). Phenoxyethanol 0.8 (11). White Clay 25.0 (12). Bentonite clay 10.0 Procedure: Mix all ingredients and heat at 60 to 70 C. Cool to room temperature. An off-white thick “mud pack” paste is obtained. It is applied on the face like any common “mud pack” mask.

### Example 8

A Moisturizing Mask “Leave-in” Delivery System Composition with Skin Lightening, Age-spot Removing, and Double-Chin Reduction Ingredients Ingredient % (1). Deionized Water 41.5 (2). Phenoxyethanol 0.7 (3). Methyl Paraben 0.2 (4). Canadian Willowherb 1.1 (5). Chamomile Extract 1.0 (6). Ascorbyl glucosamine 0.1 (7). Micromerol 0.0001 (8). Helioxine 0.0001 (9). Chlorelline 0.0001 (10). Melarrest-L 0.0001 (11). Green Tea Extract 0.0001 (12). Grapeseed Extract 0.0001 (13). Vitamin E Acetate 0.0001 (14). Lipoic Acid 0.0001 (15). Dimethiconol 2.0 (16). Silicone Wax 2.0 (17). Glycerin 50.0 (18). Xanthan Gum 1.0 (19). Aloe Vera 0.2 (20). Fragrance 0.2 Procedure: The carrier base is first made in the standard manner. All other ingredients are then added to the base at room temperature with mixing. A clear gel-like composition is obtained. It is applied on the face and neck area with fingers as a thin film. It is left on the skin to be fully absorbed. It is not rinsed off.

### Example 9

A Two-phase Treatment Mask with Antiaging and Cheek Fat reducing ingredients that requires only Water for Activation.

Ingredient % 1. Psyllium husk powder 38.0 (2). Aloe Vera Powder 0.5 (3). Xanthan Gum8.0 (4). Sea Kelp (Spirulina) Powder 3.0 (5). Preservative 0.5 (6). Glycerin 48.0 (7). Garcinia Cambogia extract 2.0 Procedure: Mix all ingredients to a light green thin paste. For product application, this product is mixed with five to ten parts of water. The paste immediately absorbs water, swells in volume, and turns into a gel. This gel is applied to face and neck area, then rinsed off after 15 minutes.

### Example 10

Anhydrous Non-Foaming Cleanser Exfoliating Mask with Ingredients for Collagen-boosting and Skin Surface Smoothing Benefits Ingredient % (1) Dimethicone 29.0 (2) Cyclomethicone 20.0 (3) Polysorbate-20 21.5 (4) Alkyl Benzoate 11.0 (5) Colloidal Oat Protein 5.0 (6) Polysorbate-805.0 (7) Vitamin A Palmitate 0.5 (8) Vitamin E Acetate 1.0 (9) Ascorbic Acid 5.5 (10) Glycolic Acid 1.0 (10) Malic Acid 0.5 Procedure. All ingredients are mixed together to a paste form. It is applied on face and neck and left for 5 to 15 minutes. It is then rubbed-off with fingers to remove dead skin cells, skin debris, skin surface oil, and other skin cleanser and exfoliating actions.

### Example 11

Clay-based Mask Composition with Deep Pore Refining, Anti-wrinkle, and Eye-zone fat (“puffy eyes”) Reduction Ingredients.

Ingredient % (1) Glycerin 39.0 (2) Calcium Sulfate 57.0 (3) Bentone Clay 0.1 (4) Preservative 0.7 (5) Dimethicone 2.0 (6) Phaseolamine 2.0 (7) Caffeine 0.5 (8) Algae Extract 0.0001 (9) Chamomile Extract 0.0001 (10) Vitamin A Palmitate 0.1 (11) Vitamin E Acetate 0.1 (12) Niacinamide Ascorbate 0.10 (13) Fragrance 1.00 Procedure. All ingredients are mixed together to a paste form. It is applied on the face and neck with fingers as a thick coating. It is left to provide benefits for 10 to 15 minutes, then rinsed off with water.

### Example 13

A Soak-in Mask Composition with Ingredients for Skin Whitening and Skin Fat Reduction.

Ingredient % (1) PEG-665.8 (2) Ammonium Acryloyldimethyltaurate/VP Copolymer 0.5 (3) Deionized water 15.0 (4) Ascorbic acid 2.0 (5) Hydroquinone USP 4.0 (6) Hydroxycitric acid 2.0 (7) Preservative 0.3 (8) Polydimethylsiloxane 10.0 (9) Lactic acid 0.1 Procedure. All of the ingredients are mixed. The mixture is heated and stirred at 60 to 70 degrees C. until the mixture is homogenous, i.e., for about five to ten minutes. The homogeneous mixture is cooled to room temperature. A thin lotion is produced. It is soaked on a pre-cut paper or fabric, which is then placed on face for 15 minutes, then removed. No rinsing is necessary at this point.

### Example 14

Alginic Acid Two-part Peel-off Facial Mask for with Ingredients for Skin Whitening, Wrinkle reduction, and Reduction of Fatty Tissue in Puffy Eyelids.

Ingredient % (1). Sodium Alginate 15.0 (2). Calcium Sulfate 5.0 (3). PEG-665.9 (4). Forskohlin 1.0 (5). Tetrahydrocurcumin 0.1 (6). Licorice Root Extract 0.5 (7). Calcium Hydroxycitrate 5.0 (8). Ascorbic acid 5.0 (9). Oat Protein 5.0 (10). Preservatives 0.5 Procedure. Mix 1 to 6 to a paste. Mix separately 7 to 10. Add to main batch and mix to a thin paste. This paste is mixed with five parts of water before application to skin. When mixed with water, the paste turns to a thick gel. Calcium ions replace sodium ions in sodium alginate, and calcium ions also bind with ascorbic and Hydroxycitric acids to fomr the gel. This gel is applied to skin. After 15 minutes, the mask is peeled-off with fingers. It comes off as a plastic-like film.

### Example 15

Peel-off Mask Composition with Skin Toning and Collagen-boosting Ingredients % (1) Sodium Alginate 15.0 (2) Polyvinyl alcohol 10.0 (3) Calcium Hydroxycitrate 2.0 (4) Calcium Sulfate 1.0 (5) Glycerin 56.4994 (6) Xanthan Gum 0.5 (7) Ascorbic acid 10.0 (8) Niacinamide 5.0 (9) Extract of Rosemary 0.0001 (10) Extract of Marigold 0.0001 (11) Extract of Sage 0.0001 (12) Extract of Ginseng 0.0001 (13) Extract of St. Johns-wart 0.0001 (14) Extract of Ruscus 0.0001 Procedure. Mix polyvinyl alcohol and glycerin and heat at 60 to 70 C to a solution. Add all other ingredients and mix. Cool to room temperature. For product application, mix one part of composition with five parts of water. A gel is formed. It is applied to face and neck areas. After 15 minutes, it is peed-off with fingers.

### Example 16

A Self-swelling Mask in Anhydrous Base. This is the composition for the first compartment of a two-compartment mask. The second compartment is water. When The contents of first compartment are mixed with five to ten parts of the contents of second compartment, then the mask composition swells in volume due to hydration of first compartment. This also releases any water-sensitive ingredients that may be in the composition of first compartment.

Ingredients % (1) Psyllium 15.0 (2) Chitosan 5.0 (3) Ascorbic acid 5.0 (4) Niacinamide 0.5 (6) Preservative 0.5 (7) Glycerin 73.5 (8) Salicylic acid 0.5 Procedure: Mix all ingredients. A light brown paste is obtained. It is mixed with five to 10 parts of water just prior to use. Upon such mixing with water, the mixture swells up in volume significantly, also releasing any water sensitive ingredients in their active form.

I claim:

1) A cosmetic mask composition suitable for facial or body application comprising:
(i) at least one skin beneficial cosmetic or drug composition, and
(ii) at least one composition to promote excess fat reduction, cellulite control, or muscle toning, or combinations thereof, and
(iii) at least one binder composition that binds with the ingredients of the compositions in (i) and (ii) by electrostatic, atomic, or ionic charges to synergistically enhance the benefits of (i) and (ii), and,
(iv) a cosmetically or pharmaceutically acceptable delivery system, or a carrier base composition.

2. A composition according to claim 1 wherein skin beneficial cosmetic or drug composition is selected to provide treatment of skin aging, skin wrinkles reduction, skin exfoliating, treatment of acne, treatment of rosacea, age-spots reduction, skin surface whitening, skin surface brightening, striae distensae (stretch marks) reduction, treatment of pimples, treatment of skin infections and lesions, varicose and spider veins reduction, blood microcirculation improvement, UVA/UVB protection of skin, skin redness reduction benefits, or combinations thereof.

3. A composition according to claim 1 wherein the composition to promote excess fat reduction, cellulite control, or toning benefits is selected from the group consisting of Forskohlin extract (from Coleus forskohlii plant), Hydroxycitric acid, (from Garcinia cambogia, and plants of Garcinia family), L-Carnitine, Creatine, Human growth hormone (HGH), Chromium picolinate, Kola seed extract, Caffeine, Niacinamide, Psyllium husk, Chitosan, Lipoprotein complexes, Polyphenols, Gymnemic acid, Pyruvic acid and Pyruvate salts, salts of Hydroxycitric acid, Phaseolamin (from Phaseolus vulgaris extract), DHEA, Chitosan, Theophylline, Theobromine (or salts thereof such as Aminophylline), Roselle tea extract, Arabinose, Inosine, Adenosine, Fructose-1,6-diphosphate, Adenosine triphosphate (ATP), Adenosine diphosphate (ADP), Indomethacin, Baicalein, Extract of the plant of genus Tephrosia, Natriuretic peptide, Laminaria extract, Extract from berries of Panax genus plant, Gymnema sylvestre extract, 9-cis, 11-trans Conjugated linoleic acid and 10-trans, 12-cis conjugated linoleic acid isomers (conjugated linoleic acid, CLA), Synephrine, Hordenine, Octopamine, Tyramine, N-Methyltyramine, Azaftig, Extract of Climbing ivy (Hedera helix), Extract of Arnica (Arnica montana), Extract of Rosemary (Rosmarinus officinalis), Extract of Marigold (Calendula officinalis), Extract of Sage (Salvia officinalis), Extract of Ginseng (Panax ginseng), Extract of St. Johns-wart (Hypericum perforatum), Extract of Ruscus (Ruscus aculeatus), Extract of meadowsweet (Filipendula ulmaria), Extract of Orthosiphon (Ortosifon stamincus), and combinations thereof.

4. A composition according to claim 1 wherein the electrostatic, atomic, or ionic charged binder composition is selected from chitosan, psyllium husk, algin, agar, carrageenan, gelatin, pectin, locust bean gum, gum arabica, xanthan gum, gellan gum, purified seaweeds (granulated Spirulina), alginate salts, rice bran husk, oat flour, oat protein, colloidal oat protein, soya flour, soya protein, wheat flour, wheat protein, milk powder, milk protein, egg powder, egg protein, casein, rice flour, corn starch, modified starches, rice starch, tapioca starch, inulin, hydrolyzed inulin, soya fibers, cotton fibers, cellulose, modified celluloses, sugars, modified carbohydrates, fenugreek fibers, silk fibers, various clays, zeolites, anhydrous zeolites, fumed silica, porous silica, alumina, various plant gums, and combinations thereof.

5. A composition according to claim 1 wherein a cosmetically acceptable delivery system or a carrier base can be selected in the form of a lotion, cream, gel, spray, thin liquid, body splash, mask, serum, solid cosmetic stick, lip balm, shampoo, liquid soap, bar soap, bath oil, cologne, hair conditioner, salve, collodion, impregnated patch, impregnated strip, skin surface implant, and any other such cosmetically or pharmaceutically acceptable topical delivery forms.

6. A composition according to claim 2 wherein the cosmetic or drug ingredient that is selected from a group consisting of antioxidants, collagen and elastin synthesis boosters, various hydroxy acids (alpha hydroxy acids, beta hydroxy acids, and polyhydroxy acids), vitamins, hormones, skin whitening agents, UVA/UVB sunscreens, antimicrobial agents, antifungal agents, blood microcirculation improvement agents (vasodilatory or vasoconstrictive), skin protectant drug actives, and combinations thereof. The quantities of such compositions can be safe and effective amounts as needed, and not limited to any specific limits.

7. A composition according to claim 5 wherein a cosmetically acceptable delivery system can be prepared separately and all other ingredients according to claim 1 can be added separately in the form of mixtures, solutions, suspensions, or emulsions to facilitate the manufacturing of such compositions.

8. A composition according to claim 6 wherein the composition to promote collagen and elastin in the skin is selected from Ascorbic acid, Ascorbic acid derivatives, Glucosamine ascorbate, Arginine ascorbate, Lysine ascorbate, Glutathione ascorbate, Nicotinamide ascorbate, Niacin ascorbate, Allantoin ascorbate, Creatine ascorbate, Creatinine ascorbate, Chondroitin ascorbate, Chitosan ascorbate, DNA Ascorbate, Carnosine ascorbate, Vitamin E, various Vitamin E derivatives, Tocotrienol, Rutin, Quercetin, Hesperedin (Citrus sinensis), Diosmin (Citrus sinensis), Mangiferin (Mangifera indica), Mangostin (Garcinia mangostana), Cyanidin (Vaccinium myrtillus), Astaxanthin (Haematococcus algae), Lutein (Tagetes patula), Lycopene (Lycopersicum esculentum), Resveratrol (Polygonum cuspidatum), Tetrahydrocurcumin (Curcuma longa), Rosmarinic acid (Rosmarinus officinalis), Hypericin (Hypericum perforatum), Ellagic acid (Punica granatum), Chlorogenic acid (Vaccinium vulgaris), Oleuropein (Olea europaea), α-Lipoic acid, Niacinamide lipoate, Glutathione, Andrographolide (Andrographis paniculata), Carnosine, Niacinamide, Potentilla erecta extract, Polyphenols, Grapeseed extract, Pycnogenol (Pine Bark extract), and combinations thereof. The quantities of such compositions can be safe and effective amounts as needed, and not limited to any specific limits.

9. A composition according to claim 6 wherein the skin beneficial hydroxy acid is selected from the group consisting of salicylic acid, lactic acid, glycolic acid, malic acid, mandelic acid, ascorbic acid, ascorbyl phosphoric acid, hydroxycitric acid, hydroxytetronic acid, citric acid, aleuritic acid, ellagic acid, rosmarinic acid, chlorogenic acid, polysulfonic acid, and hyaluronic acid (HYA). The quantities of such compositions can be safe and effective amounts as needed, and not limited to any specific limits.

10. A composition according to claim 6 wherein the skin beneficial skin whitening agent is selected from hydroquinone, arbutin, hydroquinone derivatives, Paper Mulberry extract (Broussonetia kazinoke), Mitracarpe extract (Mitracarpus scaber), Bearberry extract (Arctostaphylos uva ursi), Yellow Dock extract (Rumex crispus and Rumex occidentalis), Glutathione, Leucocyte extract, Aspergillus orizae extract (Aspergillus orizae), Licorice Root extract (Glycyrrhiza glabra), Rosmarinic acid (Rosmarinus officinalis), Tetrahydrocurcumin, Green Tea extract (Camellia sinensis), Yohimbe extract (Pausinystalia yohimbe), Ecklonia cava extract, niacinamide, Hydroxytetronic acid, Spondias mombin extract, Maprounea guianensis extract, Walteria indica extract, Gouania blanchetiana extract, Cordia schomburgkii extract, Randia armata extract, Hibiscus furcellatus extract, and combinations thereof. The quantities of such compositions can be safe and effective amounts as needed, and not limited to any specific limits.

11. A composition according to claim 6 wherein the skin beneficial antioxidant composition is selected from Ascorbic acid, Ascorbic acid derivatives, Vitamin E, Vitamin E derivatives, Tocotrienol, Rutin, Quercetin, Hesperedin (Citrus sinensis), Diosmin (Citrus sinensis), Mangiferin (Mangifera indica), Mangostin (Garcinia mangostana), Cyanidin (Vaccinium myrtillus), Astaxanthin (Haematococcus algae), Lutein (Tagetes patula), Lycopene (Lycopersicum esculentum), Resveratrol (Polygonum cuspidatum), Tetrahydrocurcumin (Curcuma longa), Rosmarinic acid (Rosmarinus officinalis), Hypericin (Hypericum perforatum), Ellagic acid (Punica granatum), Chlorogenic acid (Vaccinium vulgaris), Oleuropein (Olea europaea), alpha-Lipoic acid, Glutathione, Andrographolide, Grapeseed extract, Green Tea Extract, Polyphenols, Pycnogenol (Pine Bark extract), White Tea extract, Black Tea extract, (Andrographis paniculata), Carnosine, Niacinamide, Emblica extract, and combinations thereof. The quantities of such compositions can be safe and effective amounts as needed, and not limited to any specific limits.

12. A composition according to claim 6 wherein the UVA/UVB sunscreen composition is selected from Titanium dioxide, Zinc oxide, Galanga extract (Kaempferia galanga), Benzophenone-3, Benzophenone-4, Ethylhexyl Methoxycinnamate, Homosalate, Ethylhexyl salicylate, Octocrylene, Menthyl anthranilate, Avobenzone, Lawsone, Sulisobenzone, Trolamine salicylate, Lawsone, Glyceryl aminobenzoate, Cinoxate, and PABA, and combinations thereof. The quantities of such compositions can be safe and effective amounts as needed, and not limited to any specific limits.

13. A Composition According to claim 6 wherein the blood microcirculation improvement composition is selected from Horse Chestnut Extract (Aesculus hippocastanum extract)), Esculin, Escin, Yohimbine, Capsicum Oleoresin, Capsaicin, Niacin, Niacin Esters, Methyl Nicotinate, Benzyl Nicotinate, Ruscogenins (Butchers Broom extract; Ruscus aculeatus extract), Diosgenin (Trigonella foenumgraecum, Fenugreek), Emblica extract (Phyllanthus emblica extract), Asiaticoside (Centella asiatica extract), Boswellia Extract (Boswellia serrata), Ginger Root Extract (Zingiber Officianalis), Piperine, Vitamin K, Melilot (Melilotus officinalis extract), Glycyrrhetinic acid, Ursolic acid, Sericoside (Terminalia sericea extract), Darutoside (Siegesbeckia orientalis extract), Amni visnaga extract, extract of Red Vine (Vitis-Vinifera) leaves, apigenin, phytosan, luteolin, and combinations thereof. The quantities of such compositions can be safe and effective amounts as needed, and not limited to any specific limits.

14. A composition according to claim 6 wherein the antimicrobial composition is selected from Berberine, Triclosan, Triclocarban, various Tritons (quaternary ammonium compounds), Benzyl Alcohol, Dehydroacetic Acid, Phenoxyethanol, and combinations thereof. The quantities of such compositions can be safe and effective amounts as needed, and not limited to any specific limits.

15. A composition according to claim 6 wherein the vitamin composition is selected from Vitamin A, members of Vitamins B group, Vitamin C, Vitamin D, Vitamin E, Vitamin K, Carotenes, Biotin, Folic Acid, and combinations thereof. The quantities of such ingredients can be safe and effective amounts as needed, and not limited to any specific limits.

16. A composition according to claim 6 wherein the hormone composition is selected from progesterone, androsterone, dehydroepiandrosterone (DHEA), Pregnenolone, androstenedione, melatonin, testosterone, and combinations thereof. The quantities of such compositions can be safe and effective amounts as needed, and not limited to any specific limits.

17. A composition according to claim 6 wherein the skin protectant drug composition is selected from Allantoin, petrolatum, glycerin, dimethicone, urea, calamine, cocoa butter, kaolin, zinc acetate, zinc carbonate, and combinations thereof. The quantities of such compositions can be safe and effective amounts as needed, and not limited to any specific limits.

18. The compositions according to claim 7 wherein the cosmetically or pharmaceutically acceptable delivery system can be traditional water and oil emulsions, suspensions, colloids, microemulsions, clear solutions, suspensions of nanoparticles, emulsions of nanoparticles, or anhydrous compositions.

19. A composition according to claim 1 8 wherein cosmetically or pharmaceutically acceptable delivery system or carrier base can optionally include additional skin beneficial ingredients selected from skin cleansers, surfactants (cationic, anionic, non-ionic, amphoteric, and zwitterionic), skin and hair conditioning agents, vitamins, hormones, minerals, plant extracts, antiinflammatory agents, concentrates of plant extracts, emollients, moisturizers, skin protectants, humectants, silicones, skin soothing ingredients, analgesics, skin penetration enhancers, solubilizers, moisturizers, emollients, anesthetics, colorants, perfumes, preservatives, seeds, broken seed nut shells, silica, clays, beads, luffa particles, polyethylene balls, mica, pH adjusters, processing aids, and combinations thereof. The quantities of such ingredients can be safe and effective amounts as needed, and not limited to any specific limits.

20. A composition according to claim 18 wherein the mask may be a two-compartment composition. The composition in the first compartment can be anhydrous pr water-based. The composition in the second compartment can similarly be anhydrous or water-based.
